(19) United States
(12) Patent Application Publication
(10) Pub. No.: US 2009/0075252 A1
(43) Pub. Date: Mar. 19, 2009

(54) Methods for increasing accuracy of nucleic acid sequencing

(75) Inventors: Timothy D. Harris, Toms River, NJ (US); Eric S. Lander, Cambridge, MA (US)

Correspondence Address: EDWARDS ANGELL PALMER & DODGE LLP, P.O. BOX 55874, BOSTON, MA, 02205, US

(73) Assignee: Helicos BioSciences Corporation, Cambridge, MA (US)

(21) Appl. No.: 11/640,124

(22) Filed: Dec. 15, 2006

Related U.S. Application Data
(63) Continuation of application No. 11/404,675, filed on Apr. 14, 2006, now abandoned.

(51) Int. Cl. C12Q 1/68

(52) U.S. Cl. 435/6

(57) ABSTRACT

The invention provides methods for improving the fidelity of a sequencing-by-synthesis reaction by resequencing at least a portion of a nucleic acid template.

### RELATED APPLICATION

This application is a continuation of U.S. application Ser. No. 11/404,675 filed Apr. 14, 2006, pending, the entire contents of which is expressly incorporated herein by reference.

### TECHNICAL FIELD OF THE INVENTION

The invention generally relates to methods for increasing accuracy in nucleic acid synthesis reactions.

### BACKGROUND OF THE INVENTION

The accuracy of template-dependent nucleic acid synthesis depends in part on the ability of the polymerase to discriminate between complementary and non-complementary nucleotides. Normally, the conformation of the polymerase enzyme favors incorporation of the complementary nucleotide. However, there is still an identifiable rate of misincorporation that depends upon factors such as local sequence and the base to be incorporated.

In addition, synthetic or modified nucleotides and analogs, such as labeled nucleotides, tend to be incorporated into a primer less efficiently than naturally-occurring nucleotides. The reduced efficiency with which the unconventional nucleotides are incorporated by the polymerase can adversely affect the performance of sequencing techniques that depend upon faithful incorporation of such unconventional nucleotides.

Single molecule sequencing techniques allow the evaluation of individual nucleic acid molecules in order to identify changes and/or differences affecting genomic function. Single molecule sequencing techniques can be conducted using nucleic acid fragments as templates. Sequencing events are detected and correlated to the individual strands. See Braslavsky et al., Proc. Natl. Acad. Sci., 100: 3960-64 (2003), incorporated by reference herein. Because single molecule techniques do not rely on ensemble averaging as do bulk techniques, errors due to misincorporation can have a significant deleterious effect on the sequencing results. The incorporation of a nucleotide that is incorrectly paired, under standard Watson and Crick base-pairing, with a corresponding template nucleotide during primer extension may result in sequencing errors. The presence of misincorporated nucleotides also may result in prematurely terminated strand synthesis, reducing the number of template strands for future rounds of synthesis, and thus reducing the efficiency of sequencing.

There is, therefore, a need in the art for improved methods for reducing the frequency of misincorporation and improving the accuracy of nucleic acid synthesis reactions, especially in single molecule sequencing.

### SUMMARY OF THE INVENTION

The invention addresses the problem of misincorporation in nucleic acid synthesis reactions. The invention improves the accuracy of nucleic acid synthesis reactions by resequencing at least a portion of the template. Resequencing the template is expected to increase the accuracy of the sequence information obtained from a given template by providing more than one set of sequence information to compare, for example, to a reference sequence. In addition, the sequence information initially compiled during sequencing can be compared to the sequence information obtained from the resequencing steps to determine the accuracy of the sequencing steps.

According to the present invention, a polymerization reaction is conducted on a nucleic acid duplex that comprises a primer hybridized to a template nucleic acid. The reaction is conducted in the presence of a polymerase, and at least one nucleotide comprising a detectable label. In some embodiments, a plurality of primers is hybridized to the template at a plurality of regions of the template.

In a single molecule sequencing-by-synthesis reaction, one or more primer/template duplexes are bound to a solid support such that a least a portion of the duplexes are individually optically detectable. According to the invention, a primer/template duplex is exposed to a polymerase, and at least one detectably labeled nucleotide under conditions sufficient for template dependent nucleotide addition to the primer (also referred to herein as the polymerization reaction). Unincorporated labeled nucleotides are optionally washed away. The incorporation of the labeled nucleotide is determined, as well the identity of the nucleotide that is complementary to a nucleotide on the template at a position that is opposite the incorporated nucleotide. The polymerization reaction, optional washing and identification steps can be serially repeated in the presence of detectably labeled nucleotide that corresponds to each of the other nucleotide species. The polymerization reaction, optional washing and identification steps can be repeated a desired number of times, for example until a sequence of incorporated nucleotides is compiled from which the sequence of the template nucleic acid can be determined.

After repeating the polymerization reaction, optional washing and identification steps as described above, the primer can be removed from the duplex. The primer can be removed by any suitable means, for example by raising the temperature of the surface or substrate such that the duplex is melted, or by changing the buffer conditions to destabilize the duplex, or combination thereof. Methods for melting template/primer duplexes are well known in the art and are described, for example, in chapter 10 of Molecular Cloning, a Laboratory Manual, 3^{rd} Edition, J. Sambrook, and D. W. Russell, Cold Spring Harbor Press (2001), the teachings of which are incorporated herein by reference. The primer can then be removed from the surface, for example by rinsing the surface with a suitable rinsing solution.

After removing the primer, the template can be exposed to a second primer capable of hybridizing to the template. In one embodiment, the second primer is capable of hybridizing to the same region of the template as the first primer (also referred to herein as a first region), to form a template/primer duplex. The polymerization reaction, optional washing and identification steps can then be repeated, thereby resequencing at least a portion of the template. In one embodiment, the first and second primers have the same sequence. In another embodiment, the first and second primers have different sequences.

After repeating the polymerization reaction, optional washing and identification steps to resequence at least a portion of the template, the second primer (or primers) can be removed from the duplex as described above, and the template can be exposed to another primer capable of hybridizing to the template as described above, to form a template/primer duplex. The polymerization reaction, optional washing and identification steps can then be repeated again thereby resequencing at least a portion of the template.

In one embodiment, a plurality of primers can be hybridized to a plurality of regions on the template. During the polymerization reaction, optional washing and identification steps, sequence information is obtained from one or more of the primers. After repeating the polymerization reaction, optional washing and identification steps, the primers can be removed as described above, and a second primer or second plurality of primers can be hybridized to the template. At least one of the primers can be capable of hybridizing to the template that was previously hybridized such that at least a portion of the template can be resequenced. The template/primer duplex can comprise a plurality of primers that are hybridized to a plurality of regions on the template. In one embodiment, the first and second pluralities of primers can comprise the same sequence. In another embodiment, the first and second pluralities of primers can comprise different sequences. Sequence obtained initially and during resequencing can be analyzed and/or compared as described herein.

Single molecule sequencing methods of the invention preferably comprise template/primer duplex attached to a surface. Individual nucleotides added to the surface comprise a detectable label—preferably a fluorescent label. Each nucleotide species can comprise a different label, or can comprise the same label. In a preferred embodiment, at least a portion of each duplex is individually optically resolvable in order to facilitate single molecule sequence discrimination. The choice of a surface for attachment of duplex depends upon the detection method employed. Preferred surfaces for methods of the invention include epoxide surfaces and polyelectrolyte multilayer surfaces, such as those described in Braslavsky, et al., supra. Surfaces preferably are deposited on a substrate that is amenable to optical detection of the surface chemistry, such as glass or silica.

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine.

Polymerases useful in the invention include any nucleic acid polymerase capable of catalyzing a template-dependent addition of a nucleotide or nucleotide analog to a primer. Depending on the characteristics of the target nucleic acid, a DNA polymerase, an RNA polymerase, a reverse transcriptase, or a mutant or altered form of any of the foregoing can be used. According to one aspect of the invention, a thermophilic polymerase is used, such as ThermoSequenase®, 9°N™, Therminator™, Taq, Tne, Tma, Pfu, Tfl, Tth, Tli, Stoffel fragment, Vent™ and Deep Vent™ DNA polymerase.

### BRIEF DESCRIPTION OF THE DRAWINGS

FIG. 1 is a schematic representation of one embodiment of the present invention.

FIG. 2 is a schematic representation of another embodiment of the present invention.

FIG. 3 is a bar graph showing missing base analysis from a first and second round of sequencing of a template.

### DETAILED DESCRIPTION

The invention provides methods and compositions for improving the accuracy of a nucleic acid sequencing-by-synthesis reaction by resequencing a least a portion of the nucleic acid template. While applicable to bulk sequencing methods, the invention is particularly useful in connection with single molecule sequencing methods. Resequencing the template can increase the accuracy of the sequence information obtained from a given template by providing more than one set of sequence information to compare, for example, to a reference sequence. For example, the sequence information initially compiled during sequencing can be compared to the sequence information obtained from the resequencing steps to determine the accuracy of the sequencing steps. In some embodiments, a portion of the template can be resequenced at least once, at least three times, at least five times, at least 10 times, and at least 100 times. Likewise, the sequence information compiled during resequencing can be compared to the initial sequencing (or a reference sequence) at least once, at least three times, at least five times, at least 10 times and at least 100 times.

The present invention comprises the steps of exposing a duplex comprising a template and a primer to a polymerase and one or more nucleotide comprising a detectable label under conditions sufficient for template-dependent nucleotide addition to said primer. The primer is hybridized to a first region of the template. Any unincorporated labeled nucleotide can be washed way. Any nucleotide incorporated into the primer is identified by detecting the label associated with the incorporated nucleotide. The template/primer duplex is exposed to polymerase and another nucleotide comprising a detectable label and the polymerization reaction, optional washing and identification steps, are repeated, thereby determining a nucleotide sequence. The primer is then removed from the template, and the template is exposed to a second primer capable of hybridizing to the first region of the template to form a template/primer duplex. The steps of exposing the template primer duplex to polymerase and nucleotide comprising a detectable label, optional washing and identification can be conducted to thereby resequence a portion of the template, thereby increasing the accuracy of nucleic acid sequencing. In one embodiment, a plurality of primers is hybridized to a plurality of regions on the template. According to the invention, the template, primer and/or the duplex can be labeled such that it is individually optically resolvable.

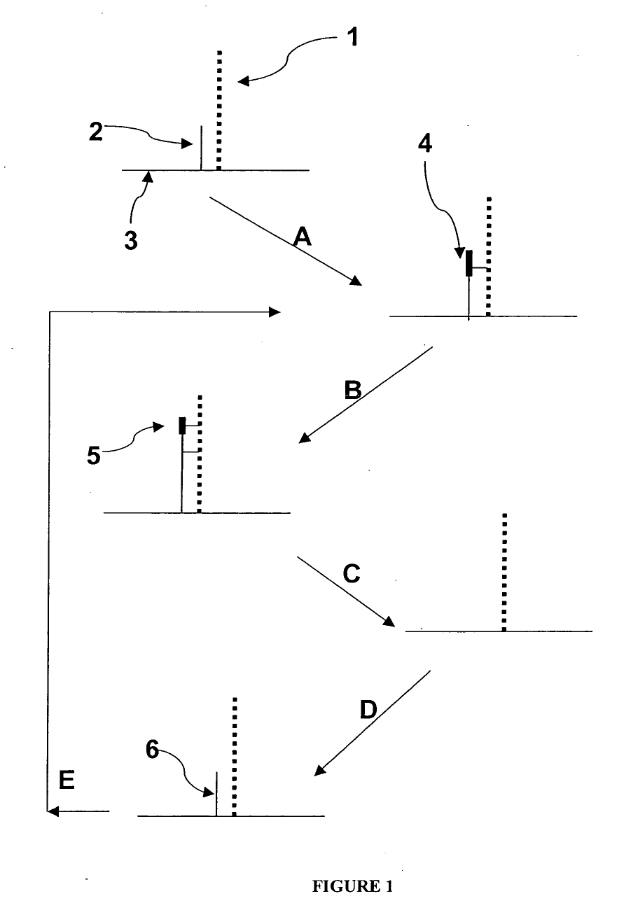

FIG. 1 is a schematic representation of one embodiment of the present invention. In this embodiment, a nucleic acid template, 1, is attached to a solid support, 3. A primer, 2, is hybridized to the template, forming a template/primer duplex. In step A, the template primer duplex is exposed to a polymerase and at least one nucleotide comprising a detectable label under conditions sufficient for template-dependent nucleotide addition to said primer. If the nucleotide is complementary to the template nucleotide immediately downstream of the primer, a nucleotide, 4 is added to the primer. After identifying nucleotide incorporated into said primer, the process is repeated in step B, thereby adding a second nucleotide to the primer in a template dependent manner. After the process has been repeated the desired number of times, the primer is removed as shown in step C. In step D, a primer, 6, is hybridized to the template, forming a template/primer duplex. The process of adding nucleotide and polymerase, detecting incorporated nucleotide and repeating the desired number of times is then repeated as shown in step E.

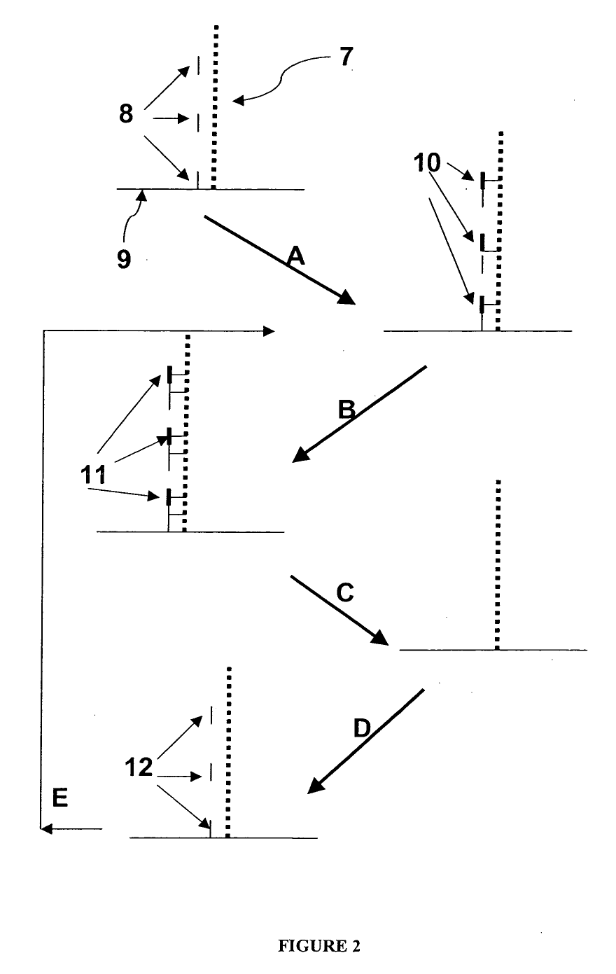

FIG. 2 is a schematic representation of another embodiment of the present invention. In this embodiment, a nucleic acid template, 7, is attached to a solid support, 9. A plurality of primers, 8, is hybridized to the template at a plurality of regions, forming a template/primer duplex. In step A, the template primer duplex is exposed to a polymerase and at least one nucleotide comprising a detectable label under conditions sufficient for template-dependent nucleotide addition to the plurality of primers. If the nucleotide is complementary to the template nucleotide immediately downstream of a primer, a nucleotide, 10 is added to the primer. After identifying nucleotide incorporated into said primer, the process is repeated in step B, thereby adding a second nucleotide to the primer in a template dependent manner. After the process has been repeated the desired number of times, the plurality of primers are removed as shown in step C. In step D, a plurality of primers, 12, is hybridized to the template at a plurality of regions, forming a template/primer duplex. The process of adding nucleotide and polymerase, detecting incorporated nucleotide and repeating the desired number of times is then repeated as shown in step E.

Methods and compositions of the invention are well-suited for use in single molecule sequencing techniques. Substrate-bound primer/template duplexes are exposed to a polymerase and at least one labeled nucleotide corresponding to a first nucleotide species. The duplexes are washed of unincorporated labeled nucleotides, and the incorporation of labeled nucleotide is determined. The identity of the nucleotide positioned on the template opposite the incorporate nucleotide is likewise determined. The polymerization reaction is serially repeated in the presence of a labeled nucleotide that corresponds to each of the other nucleotide species in order to compile a sequence of incorporated nucleotides that is representative of the complement to the template nucleic acid.

In a preferred embodiment of the invention, direct amine attachment is used to attach primer, template, or both as duplex to an epoxide surface. The primer or the template comprises an optically-detectable label in order to determine the location of duplex on the surface. At least a portion of the duplex must be optically resolvable from other duplex on the surface. The surface is preferably passivated with a reagent that occupies portions of the surface that might, absent passivation, fluoresce. Optimal passivation reagents include amines, phosphate, water, sulfates, detergents, and other reagents that reduce native or accumulating surface fluorescence. Sequencing is then accomplished by presenting one or more labeled nucleotide in the presence of a polymerase under conditions that promote complementary base incorporation in the primer. In a preferred embodiment, one base at a time (per cycle) is added and all bases have the same label. There is a wash step after each incorporation cycle, and the label is either neutralized without removal or removed from incorporated nucleotides. After the completion of a predetermined number of cycles of base addition, the linear sequence data for each individual duplex is compiled. Numerous algorithms are available for sequence compilation and alignment as discussed below.

In general, epoxide-coated glass surfaces are used for direct amine attachment of templates, primers, or both. Amine attachment to the termini of template and primer molecules is accomplished using terminal transferase. Primer molecules can be custom-synthesized to hybridize to templates for duplex formation.

A full-cycle is conducted as many times as necessary to complete sequencing of a desired length of template. Once the desired number of cycles is complete, the result is a stack of images represented in a computer database. For each spot on the surface that contained an initial individual duplex, there will be a series of light and dark image coordinates, corresponding to whether a base was incorporated in any given cycle. For example, if the template sequence was TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence of the template would be built up through the image stack. The sequencing data are then fed into an aligner as described below for resequencing, or are compiled for de novo sequencing as the linear order of nucleotides incorporated into the primer.

The imaging system to be used in the invention can be any system that provides sufficient illumination of the sequencing surface at a magnification such that single fluorescent molecules can be resolved. In general, the system comprised three lasers, one that produces “green” light, one that produces “red” light, and in infrared laser that aids in focusing. The beams are transmitted through a series of objectives and mirrors, and focused on the surface. Imaging is accomplished with an inverted Nikon TE-2000

### General Considerations

A. Nucleic Acid Templates

Nucleic acid templates include deoxyribonucleic acid (DNA) and/or ribonucleic acid (RNA). Nucleic acid template molecules can be isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples of the present invention include viral particles or preparations. Nucleic acid template molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules may also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

B. Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adeno sine, cytidine, guano sine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably comprise a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

C. Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al., 1998, Proc Natl Acad. Sci. USA 95:14250-->5).

While mesophilic polymerases are contemplated by the invention, preferred polymerases are thermophilic. Thermophilic DNA polymerases include, but are not limited to, ThermoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

D. Surfaces

In a preferred embodiment, nucleic acid template molecules are attached to a substrate (also referred to herein as a surface) and subjected to analysis by single molecule sequencing as taught herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the nucleic acid template molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

E. Detection

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

F. Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

In resequencing, a preferred embodiment for sequence alignment compared sequences obtained to a database of reference sequences of the same length, or within 1 or 2 bases of the same length, from the initially obtained sequence or the target sequence contained in a look-up table format. In a preferred embodiment, the look-up table contains exact matches with respect to the reference sequence and sequences of the prescribed length or lengths that have one or two errors (e.g., 9-mers with all possible 1-base or 2-base errors). The obtained sequences are then matched to the sequences on the look-up table and given a score that reflects the uniqueness of the match to sequence(s) in the table. The obtained sequences are then aligned to the reference sequence based upon the position at which the obtained sequence best matches a portion of the reference sequence. More detail on the alignment process is provided below in the Example.

Certain embodiments of the invention are described in the following examples, which are not meant to be limiting.

### EXAMPLE I

### Melt and Resequence Test

Approximately 20 pmol of template DNA was polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62). The average dA tail length was 50+/−5 nucleotides. Terminal transferase was then used to label the polyadenylated templates with Cy3-dUTP. Polyadenylated labeled templates were then terminated with dideoxyTTP (also added using terminal transferase). The resulting templates were filtered with a YM10 ultrafiltration spin column to remove free nucleotides and stored in ddH₂O at −20° C.

Epoxide-coated glass slides were prepared for oligo attachment. Epoxide-functionalized 40 mm diameter #1.5 glass cover slips (slides) were obtained from Erie Scientific (Salem, N.H.). The slides were preconditioned by soaking in 3×SSC for 15 minutes at 37° C. Next, a 500 pM aliquot of 5′ aminated templates described above were incubated with each slide for 30 minutes at room temperature in a volume of 80 ml. The resulting slides have poly(dA50) templates attached by direct amine linkage to the epoxide. The slides are then treated with phosphate (1 M) for 4 hours at room temperature in order to passivate the surface. Slides are then stored in polymerase rinse buffer (20 mM Tris, 100 mM NaCl, 0.001% Triton X-100, pH 8.0) until they are used for sequencing.

For sequencing, the slides were placed in a modified FCS2 flow cell (Bioptechs, Butler, Pa.) using a 50 um thick gasket The flow cell was placed on a movable stage that is part of a high-efficiency fluorescence imaging system built around a Nikon TE-2000 inverted microscope equipped with a total internal reflection (TIR) objective. The slide was then rinsed with HEPES buffer with 100 mM NaCl and equilibrated to a temperature of 50° C. A 1 nM aliquot of poly(dT50) primer in 3×SSC was placed in the flow cell and incubated on the slide for 20 minutes. After incubation, the flow cell was rinsed with 1×SSC/HEPES/0.1% SDS followed by HEPES/NaCl. A passive vacuum apparatus was used to pull fluid across the flow cell. The resulting slide contained template/oligo(dT) primer duplex. The temperature of the flow cell was then reduced to 37° C. for sequencing and the objective was brought into contact with the flow cell.

For sequencing, cytosine triphosphate, guanidine triphosphate, adenine triphosphate, and uracil triphosphate, each having a cyanine-5 label (at the 7-deaza position for ATP and GTP and at the C5 position for CTP and UTP (PerkinElmer)) were stored separately in buffer containing 20 mM Tris-HCl, pH 8.8, 10 mM (NH₄)₂SO₄, 10 mM KCl, 10 mM NaCl, 50 μM MnSO₄, and 0.1% Triton X-100, and 50 U/ml Klenow exo-polymerase (NEN). Sequencing proceeds as follows.

First, initial imaging was used to determine the positions of duplex on the epoxide surface. The Cy3 label attached to the templates was imaged by excitation using a laser tuned to 532 nm radiation (Verdi V-2 Laser, Coherent, Inc., Santa Clara, Calif.) in order to establish duplex position. For each slide only single fluorescent molecules imaged in this step were counted. Imaging of incorporated nucleotides as described below was accomplished by excitation of a cyanine-5 dye using a 635 nm radiation laser (Coherent). 250 nM Cy5CTP was placed into the flow cell and exposed to the slide for 2 minutes. After incubation, the slide was rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes). An oxygen scavenger containing 30% acetonitrile and scavenger buffer (134 ul HEPES/NaCl, 24 ul 100 mM Trolox in MES, pH 6.1, 10 ul DABCO in MES, pH 6.1, 8 ul 2M glucose, 20 ul NaI (50 mM stock in water), and 4 ul glucose oxidase) was next added. The slide was then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence were recorded. After imaging, the flow cell was rinsed 5 times each with SSC/HEPES/SDS (60 ul) and HEPES/NaCl (60 ul). Next, the cyanine-5 label was cleaved off incorporated CTP by introduction into the flow cell of 50 mM TCEP for 5 minutes, after which the flow cell was rinsed 5 times each with SSC/HEPES/SDS (60 ul) and HEPES/NaCl (60 ul). The remaining nucleotide was capped with 50 mM iodoacetamide for 5 minutes followed by rinsing 5 times each with SSC/HEPES/SDS (60 ul) and HEPES/NaCl (60 ul). The scavenger was applied again in the manner described above, and the slide was again imaged to determine the effectiveness of the cleave/cap steps and to identify non-incorporated fluorescent objects.

The procedure described above was then conducted 500 nM Cy5dUTP, followed by 250 nM Cy5dGTP, and finally 500 nM Cy5dATP. The procedure (expose to nucleotide, polymerase, rinse, scavenger, image, rinse, cleave, rinse, cap, rinse, scavenger, final image) is repeated exactly as described for ATP, GTP, and UTPs. Uridine was used instead of Thymidine due to the fact that the Cy5 label was incorporated at the position normally occupied by the methyl group in Thymidine triphosphate, thus turning the dTTP into dUTP. In all 12 cycles (C, U, A, G) were conducted as described in this and the preceding paragraph.

Once the desired number of cycles was completed, the image stack data (i.e., the single molecule sequences obtained from the various surface-bound duplex) was analyzed and compared to the known template sequence.

The primers were removed by flowing in 1 ml 70° C. diH₂O, incubating for 2 minutes and repeating 2 times with an additional 1 ml of 70° C. diH₂O each time. The surface was then imaged to confirm the removal of the primer.

An aliquot of poly(dT50) primer was placed in the flow cell and incubated as described above. After incubation, the flow cell was rinsed, and the procedure (expose to nucleotide, polymerase, rinse, scavenger, image, rinse, cleave, rinse, cap, rinse, scavenger, final image) was repeated as described above.

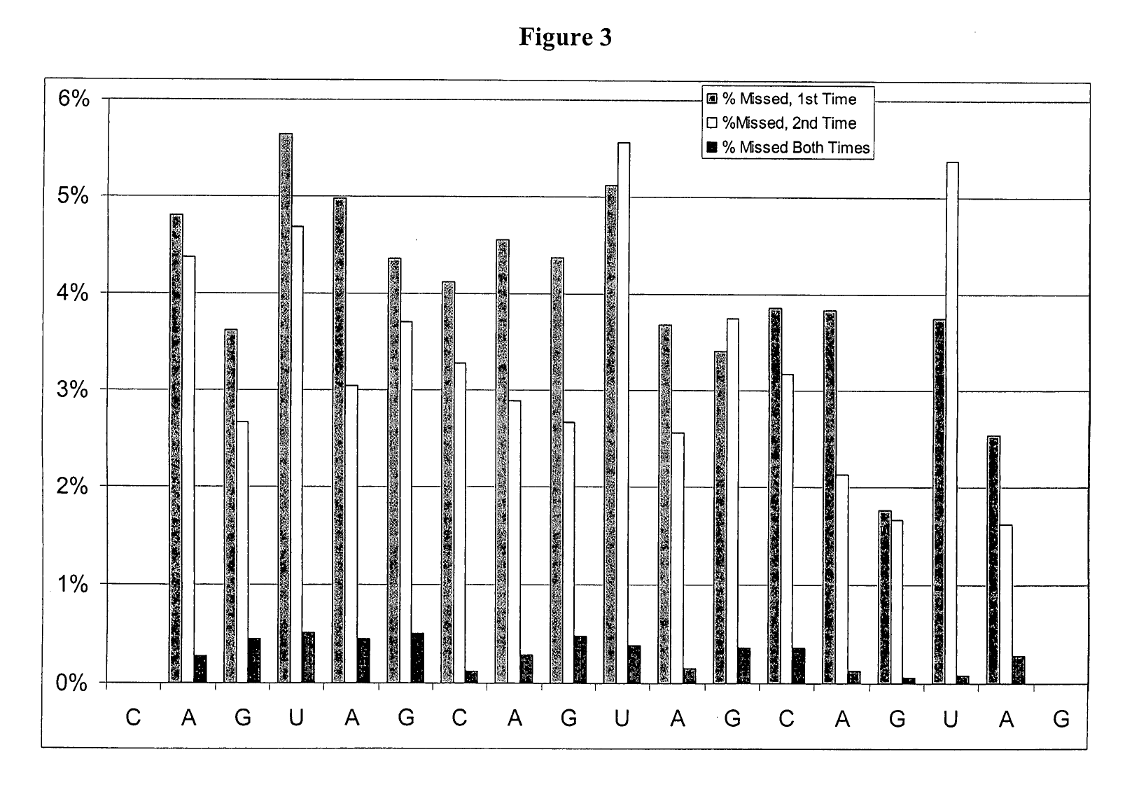

Once the desired number of cycles is completed, the image stack data (i.e., the single molecule sequences obtained from the various surface-bound duplex) are aligned to the known template sequence and/or are aligned to the sequence initially obtained as described above. Of the templates in which at least 6 nucleotides were sequenced in the first round, 93% of the duplexes were melted (resulted in the removal of the primer), 65% were melted and rehybridized, and 54% were melted, rehybridized and at least 6 nucleotides were added in the second round of sequencing. As shown in FIG. 3, in the first or second round of synthesis, a template nucleotide was mis-sequenced from about 1.7 to about 6.5% of the time. However, when the data from the first and second rounds of sequencing are compared, a given template nucleotide was mis-sequenced in both rounds of sequencing 0.1 to 0.5% of the time. Therefore, resequencing a template one time results in a 10 fold increase in accuracy.

### EXAMPLE II

The 7249 nucleotide genome of the bacteriophage M13 mp18 was sequenced using single molecule methods of the invention. Purified, single-stranded viral M13mp18 genomic DNA was obtained from New England BioLabs. Approximately 25 ug of M13 DNA was digested to an average fragment size of 40 bp with 0.1 U Dnase I (New England BioLabs) for 10 minutes at 37° C. Digested DNA fragment sizes were estimated by running an aliquot of the digestion mixture on a precast denaturing (TBE-Urea) 10% polyacrylamide gel (Novagen) and staining with SYBR Gold (Invitrogen/Molecular Probes). The DNase I-digested genomic DNA was filtered through a YM10 ultrafiltration spin column (Millipore) to remove small digestion products less than about 30 nt. Approximately 20 pmol of the filtered DNase I digest was then polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62.). The average dA tail length was 50+/−5 nucleotides. Terminal transferase was then used to label the fragments with Cy3-dUTP. Fragments were then terminated with dideoxyTTP (also added using terminal transferase). The resulting fragments were again filtered with a YM10 ultrafiltration spin column to remove free nucleotides and stored in ddH₂O at −20° C.

Glass slides were prepared and mounted on the microscope as described above. The slide is then rinsed with HEPES buffer with 100 mM NaCl and equilibrated to a temperature of 50° C. An aliquot of poly(dT50) primer is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains M13 template/oligo(dT) primer duplex. The temperature of the flow cell is then reduced to 37° C. for sequencing and the objective is brought into contact with the flow cell.

For sequencing, cytosine triphosphate, guanidine triphosphate, adenine triphosphate, and uracil triphosphate, each having a cyanine-5 label (at the 7-deaza position for ATP and GTP and at the C5 position for CTP and UTP (PerkinElmer)) are stored separately in buffer containing 20 mM Tris-HCl, pH 8.8, 10 mM MgSO₄, 10 mM (NH₄)₂SO₄, 10 mM HCl, and 0.1% Triton X-100, and 100U Klenow exo⁻ polymerase (NEN). Sequencing proceeds as follows.

First, initial imaging is used to determine the positions of duplex on the epoxide surface. The Cy3 label attached to the M13 templates is imaged by excitation using a laser tuned to 532 nm radiation (Verdi V-2 Laser, Coherent, Inc., Santa Clara, Calif.) in order to establish duplex position. For each slide only single fluorescent molecules imaged in this step are counted. Imaging of incorporated nucleotides as described below is accomplished by excitation of a cyanine-5 dye using a 635 nm radiation laser (Coherent). 5 uM Cy5CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes). An oxygen scavenger containing 30% acetonitrile and scavenger buffer (134 ul HEPES/NaCl, 24 ul 100 mM Trolox in MES, pH 6.1, 10 ul DABCO in MES, pH 6.1, 8 ul 2M glucose, 20 ul NaI (50 mM stock in water), and 4 ul glucose oxidase) is next added. The slide is then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 ul). Next, the cyanine-5 label is cleaved off incorporated CTP by introduction into the flow cell of 50 mM TCEP for 5 minutes, after which the flow cell is rinsed 5 times each with SSC/HEPES/SDS (60 ul) and HEPES/NaCl (60 ul). The remaining nucleotide is capped with 50 mM iodoacetamide for 5 minutes followed by rinsing 5 times each with SSC/HEPES/SDS (60 ul) and HEPES/NaCl (60 ul). The scavenger is applied again in the manner described above, and the slide is again imaged to determine the effectiveness of the cleave/cap steps and to identify non-incorporated fluorescent objects.

The procedure described above is then conducted 100 nM Cy5dATP, followed by 100 nM Cy5dGTP, and finally 500 nM Cy5dUTP. The procedure (expose to nucleotide, polymerase, rinse, scavenger, image, rinse, cleave, rinse, cap, rinse, scavenger, final image) is repeated exactly as described for ATP, GTP, and UTP except that Cy5dUTP is incubated for 5 minutes instead of 2 minutes. Uridine is used instead of Thymidine due to the fact that the Cy5 label is incorporated at the position normally occupied by the methyl group in Thymidine triphosphate, thus turning the dTTP into dUTP. In all 64 cycles (C, A, G, U) are conducted as described in this and the preceding paragraph.

Once the desired number of cycles is completed, the image stack data (i.e., the single molecule sequences obtained from the various surface-bound duplex) are aligned to the M13 reference sequence. The image data obtained can be compressed to collapse homopolymeric regions. Thus, the sequence “TCAAAGC” is represented as “TCAGC” in the data tags used for alignment. Similarly, homopolymeric regions in the reference sequence are collapsed for alignment.

The alignment algorithm matches sequences obtained as described above with the actual M13 linear sequence. Placement of obtained sequence on M13 is based upon the best match between the obtained sequence and a portion of M13 of the same length, taking into consideration 0, 1, or 2 possible errors. All obtained 9-mers with 0 errors (meaning that they exactly match a 9-mer in the M13 reference sequence) are first aligned with M13. Then 10-, 11-, and 12-mers with 0 or 1 error are aligned. Finally, all 13-mers or greater with 0, 1, or 2 errors are aligned.

The primers are removed by increasing the temperature of the flow cell above the melting temperature of the duplex. After raising the temperature of the flow cell to be above the melting temperature of the duplex, the primer is released from the duplex. The free primer is removed from the flow cell by washing the flow cell, for example the flow cell can be rinsed 5 times each with SSC/HEPES/SDS (60 ul) and HEPES/NaCl (60 ul).

An aliquot of poly(dT50) primer is placed in the flow cell and incubated on the slide for 15 minutes. After incubation, the flow cell is rinsed with 1×SSC/HEPES/0.1% SDS followed by HEPES/NaCl. The resulting slide contains M13 template/oligo(dT) primer duplex. The temperature of the flow cell is then reduced to 37° C. for sequencing and the objective is brought into contact with the flow cell. The procedure (expose to nucleotide, polymerase, rinse, scavenger, image, rinse, cleave, rinse, cap, rinse, scavenger, final image) is repeated as described above.

Once the desired number of cycles is completed, the image stack data (i.e., the single molecule sequences obtained from the various surface-bound duplex) are aligned to the M13 reference sequence and/or are aligned to the sequence initially obtained as described above. The image data obtained can be compressed to collapse homopolymeric regions as described above.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A method of increasing accuracy of nucleic acid sequencing, the method comprising the steps of:
a) exposing a duplex comprising a template and a primer to a polymerase and one or more nucleotide comprising a detectable label under conditions sufficient for template-dependent nucleotide addition to said primer, the primer being hybridized to a first region of the template, wherein said duplex is individually optically resolvable;
b) identifying nucleotide incorporated into said primer;
c) repeating steps a) and b), thereby determining a nucleotide sequence;
d) removing the primer from the template;
e) exposing the template to a second primer capable of hybridizing to the first region of the template to form a template/primer duplex, and repeating steps a) through c) to resequence a portion of the template, thereby increasing the accuracy of nucleic acid sequencing.

2. The method of claim 1, wherein the sequence obtained in c) is compared with the sequence obtained in e).

3. The method of claim 1, wherein the first and second primers have identical sequence.

4. The method of claim 1, wherein the first and second primers have different sequences.

5. The method of claim 1, further comprising the step of removing the primer from the template and repeating step (e) at least once.

6. The method of claim 1, wherein said label is an optically-detectable label.

7. The method of claim 6, wherein said optically-detectable label is a fluorescent label.

8. The method of claim 7, wherein said fluorescent label is selected from the group consisting of fluorescein, rhodamine, cyanine, Cy5, Cy3, BODIPY, alexa, and derivatives thereof.

9. The method of claim 1, wherein said duplex is attached to a surface.

10. The method of claim 1, wherein a plurality of primers is hybridized to a plurality of regions on said template.

11. The method of claim 10, wherein a plurality of regions are sequenced.

12. The method of claim 11, wherein a plurality of regions are resequenced.

13. A method of increasing accuracy of nucleic acid sequencing, the method comprising the steps of:
a) exposing a duplex comprising a template and a plurality of primers to a polymerase and one or more nucleotide comprising a detectable label under conditions sufficient for template-dependent nucleotide addition to at least one of said plurality of primers, the plurality of primers being hybridized to a plurality of regions of the template, wherein said duplex is individually optically resolvable;
b) identifying incorporated nucleotides;
c) repeating steps a) and b), thereby determining a nucleotide sequence of at least one of said plurality of regions of the template;
d) removing at least one of said plurality of primers from the template;
e) exposing the template to a second plurality of primers capable of hybridizing to the first region of the template to form a template/primer duplex, and repeating steps a) and c) to resequence the at least one of said plurality of regions of the template, thereby increasing the accuracy of nucleic acid sequencing.

14. The method of claim 13, wherein sequence obtained in c) is compared with sequence obtained in e).

15. The method of claim 13, wherein the first and second pluralities of primers have identical sequences.

16. The method of claim 13, wherein the first and second pluralities of primers have different sequences.

17. The method of claim 13, wherein each of the plurality of primers is removed in step d).

18. The method of claim 13, further comprising the step of removing at least one primer from the template and repeating step e) at least once.

19. The method of claim 13, wherein said label is an optically-detectable label.

20. The method of claim 18, wherein said optically-detectable label is a fluorescent label.

21. The method of claim 20, wherein said fluorescent label is selected from the group consisting of fluorescein, rhodamine, cyanine, Cy5, Cy3, BODIPY, alexa, and derivatives thereof.

22. The method of claim 13, wherein said duplex is attached to a surface.
